SEQUENCE LISTING (30 sequences)

SEQ ID NO: 1 - DNA, 23 bp, Artificial Sequence
  Description of Artificial Sequence Synthetic primer
aatgctggcctaatagacttgca

SEQ ID NO: 2 - DNA, 23 bp, Artificial Sequence
  Description of Artificial Sequence Synthetic primer
ccgtggaatatgtaacgatttgg

SEQ ID NO: 3 - DNA, 16 bp, Artificial Sequence
  Description of Artificial Sequence Synthetic primer
accccgtcaagtagtg

SEQ ID NO: 4 - DNA, 15 bp, Artificial Sequence
  Description of Artificial Sequence Synthetic primer
ccccaaagttggcgt

SEQ ID NO: 5 - DNA, 20 bp, Artificial Sequence
  Description of Artificial Sequence Synthetic primer
ggcaaattcaacggcacagt

SEQ ID NO: 6 - DNA, 20 bp, Artificial Sequence
  Description of Artificial Sequence Synthetic primer
cgctcctggaagatggtgat

SEQ ID NO: 7 - DNA, 20 bp, Artificial Sequence
  Description of Artificial Sequence Synthetic primer
atgacatcaagaaggtggtg

SEQ ID NO: 8 - DNA, 20 bp, Artificial Sequence
  Description of Artificial Sequence Synthetic primer
cataccaggaaatgagcttg

SEQ ID NO: 9 - DNA, 20 bp, Artificial Sequence
  Description of Artificial Sequence Synthetic primer
gaagggaagtgcagcaaagg

SEQ ID NO: 10 - DNA, 24 bp, Artificial Sequence
  Description of Artificial Sequence Synthetic primer
cagagatcttcactgcattggcta

SEQ ID NO: 11 - RNA, 22 nt, Homo sapiens
uucccuuugucauccuaugccu

SEQ ID NO: 12 - RNA, 23 nt, Homo sapiens
uagcaccauuugaaaucaguguu

SEQ ID NO: 13 - RNA, 23 nt, Homo sapiens
guccaguuuucccaggaaucccu

SEQ ID NO: 14 - RNA, 22 nt, Homo sapiens
ucagugcacuacagaacuuugu

SEQ ID NO: 15 - RNA, 21 nt, Homo sapiens
aucacauugccagggauuacc

SEQ ID NO: 16 - RNA, 23 nt, Homo sapiens
ucuuugguuaucuagcuguauga

SEQ ID NO: 17 - RNA, 22 nt, Homo sapiens
uggagugugacaaugguguuug

SEQ ID NO: 18 - RNA, 108 nt, Artificial Sequence
  Description of Artificial Sequence Synthetic polynucleotide
gugcucgcuucggcagcacauauacuaaaauuggaacgauacagagaagauuuagcauggccccugcgcaaggaugacacgcaaauucgugaagcguuccauauuuuu

SEQ ID NO: 19 - RNA, 77 nt, Artificial Sequence
  Description of Artificial Sequence Synthetic oligonucleotide
aaccaaugauguaaugauucugccaaaugaaauauaaugauaucacuguaaaaccguuccauuuugauucugagguu

SEQ ID NO: 20 - DNA, 15 bp, Artificial Sequence
  Description of Artificial Sequence Synthetic oligonucleotide
aggatgacaaaggga

SEQ ID NO: 21 - DNA, 15 bp, Artificial Sequence
  Description of Artificial Sequence Synthetic oligonucleotide
acgtctatacgccca

SEQ ID NO: 22 - DNA, 22 bp, Artificial Sequence
  Description of Artificial Sequence Synthetic probe
aggcataggatgacaaagggaa

SEQ ID NO: 23 - RNA, 110 nt, Homo sapiens
ggcuacagucuuucuucaugugacucguggacuucccuuugucauccuaugccugagaauauaugaaggaggcugggaaggcaaagggacguucaauugucaucacuggc

SEQ ID NO: 24 - RNA, 7 nt, Artificial Sequence
  Description of Artificial Sequence Synthetic oligonucleotide
ucccuuu

SEQ ID NO: 25 - RNA, 22 nt, Homo sapiens
uucccuuugucauccuucgccu

SEQ ID NO: 26 - RNA, 7 nt, Artificial Sequence
  Description of Artificial Sequence Synthetic oligonucleotide
agggaaa

SEQ ID NO: 27 - RNA, 21 nt, Homo sapiens
gcugggaaggcaaagggacgu

SEQ ID NO: 28 - RNA, 110 nt, Homo sapiens
ucaccuggccaugugacuugugggcuucccuuugucauccuucgccuagggcucugagcagggcagggacagcaaaggggugcucaguugucacuucccacagcacggag

SEQ ID NO: 29 - RNA, 22 nt, Homo sapiens
uucccuuugucauccuucgccu

SEQ ID NO: 30 - RNA, 21 nt, Homo sapiens
gcagggacagcaaaggggugc

(19) United States
(12) Patent Application Publication
(10) Pub. No.: US 2016/0122762 A1
(43) Pub. Date: May 5, 2016

(54) METHODS OF TREATING ATHEROSCLEROSIS

(71) Applicant: UNIVERSITY OF IOWA RESEARCH FOUNDATION, Iowa City, IA (US)

(72) Inventors: Kaikobad Irani, Iowa City, IA (US); Ajit Vikram, Iowa City, IA (US)

(73) Assignee: UNIVERSITY OF IOWA RESEARCH FOUNDATION, Iowa City, IA (US)

(21) Appl. No.: 14/924,414

(22) Filed: Oct. 27, 2015

Related U.S. Application Data
(60) Provisional application No. 62/069,121, filed on Oct. 27, 2014.

(51) Int. Cl. C12N 15/113; C12Q 1/68

(52) U.S. Cl. CPC C12N 15/113; C12Q 1/6883; C12Q 2600/178; C12Q 2600/158; C12Q 2600/136; C12N 2310/141

(57) ABSTRACT

Certain embodiments of the invention provide a method of treating endothelial dysfunction, cardiovascular disease and/or atherosclerosis in a mammal, comprising administering an effective amount of a micro-RNA-204-5p inhibitor to the mammal.

### RELATED APPLICATIONS

This application claims the benefit of priority of U.S. Provisional Application Ser. No. 62/069,121 filed on Oct. 27, 2014, which application is herein incorporated by reference.

### BACKGROUND OF THE INVENTION

Atherosclerosis is a vascular condition which initiates with the impairment in endothelial function and deposition of fat in the sub-endothelial layer, which ultimately leads to the atheromatous plaque formation and obstruction in the blood flow in arteries. Atherosclerosis is a highly prevalent condition across the world, which is further expected to rise owing to sedentary life-style and increased consumption of dietary fat. Lowering cholesterol is the only current intervention proven to reduce atherosclerosis. New approaches and compounds for the treatment of atherosclerosis, which attack the problem at its origin, i.e., the endothelium, are needed.

### SUMMARY OF THE INVENTION

In certain embodiments, the present invention provides a method of treating endothelial dysfunction, cardiovascular disease and/or atherosclerosis in a mammal, comprising administering an effective amount of a micro-RNA-204-5p inhibitor to the mammal.

In certain embodiments, the inhibitor reduces microRNA-204-5p expression or function.

In certain embodiments, miRNA-204-5p expression and/or function is reduced by at least 10%.

In certain embodiments the miRNA-204-5p inhibitor increases SIRTUIN1 (SIRT1) expression in a cell in the mammal, as compared to SIRT1 expression in a cell in a mammal that was not administered the micro-RNA-204-5p inhibitor.

In certain embodiments, the miRNA-204-5p inhibitor increases endothelial nitric oxide synthase (eNOS) expression in a cell in the mammal, as compared to eNOS expression in a cell in a mammal that was not administered the micro-RNA-204-5p inhibitor.

In certain embodiments, the miRNA-204-5p inhibitor comprises an oligonucleotide comprising 4 to 7 nucleotides that are complementary to miRNA-204-5p seed sequence 5′-UCCCUUU-3′ (SEQ ID NO:24).

In certain embodiments, the miRNA-204-5p inhibitor comprises an oligonucleotide comprising a sequence having at least about 70% complementarity to miRNA-204-5p seed sequence 5′-UCCCUUU-3′ (SEQ ID NO:24).

In certain embodiments, the miRNA-204-5p inhibitor comprises an oligonucleotide comprising a sequence having at least about 70% complementarity with miRNA-204-5p (5′-UUCCCUUUGUCAUCCUAUGCCU-3′ (SEQ ID NO:11)).

In certain embodiments the miRNA-204-5p inhibitor comprises an oligonucleotide comprising a sequence having at least about 70% sequence identity to miRNA-204-I (5′-AGGATGACAAAGGGA-3′ (SEQ ID NO:20)).

In certain embodiments the miRNA-204-5p inhibitor comprises an oligonucleotide comprising a sequence having at least about 70% complementarity to miRNA-211 (5′-UUCCCUUUGUCAUCCUUCGCCU-3′ (SEQ ID NO:25)).

In certain embodiments the oligonucleotide is between about 18 to about 25 nucleotides in length.

In certain embodiments the oligonucleotide is chemically modified.

In certain embodiments the miRNA-204-5p inhibitor comprises an miRNA sponge.

In certain embodiments the miRNA-204-5p inhibitor comprises a small molecule inhibitor.

In certain embodiments, the present invention provides a miRNA-204-5p inhibitor for use in treating endothelial dysfunction, cardiovascular disease and/or atherosclerosis in a mammal (e.g., a human).

In certain embodiments, the present invention provides a use of a miRNA-204-5p inhibitor to prepare a medicament for treating endothelial dysfunction, cardiovascular disease and/or atherosclerosis in a mammal (e.g., a human).

In certain embodiments, the present invention provides a miRNA-204-5p inhibitor for use in medical therapy.

In certain embodiments, the present invention provides a method of decreasing dysfunction in an endothelial cell that expresses micro-RNA-204-5p, comprising contacting the cell in vivo or in vitro with a micro-RNA-204-5p inhibitor.

In certain embodiments, the present invention provides a method of identifying a miRNA-204-5p inhibitor comprising comparing the level of miRNA-204-5p expression in an endothelial cell(s) before and after exposure to a small molecule, wherein the small molecule is a miRNA-204-5p inhibitor if the level of miRNA-204-5p expression is decreased in the endothelial cell(s) after exposure to the small molecule.

In certain embodiments, the present invention further comprises comparing SIRT1 expression in the endothelial cell(s) before and after exposure to the small molecule.

In certain embodiments, the level of SIRT1 expression is increased in the endothelial cell(s) after exposure to the small molecule.

In certain embodiments, the endothelial cell(s) was exposed to serum from an animal fed a high fat diet prior to exposure to the small molecule.

In certain embodiments, the present invention provides a method of identifying a miRNA-204-5p inhibitor comprising measuring a first level of miRNA-204-5p expression in an endothelial cell(s); contacting the endothelial cell(s) with a small molecule; measuring a second level of miRNA-204-5p expression in the endothelial cell(s); and comparing the first level of miRNA-204-5p expression with the second level of miRNA-204-5p expression, wherein, the small molecule is a miRNA-204-5p inhibitor if the second level of expression is decreased as compared to the first level of expression.

In certain embodiments, the method further comprises measuring a first level of SIRT1 expression in the endothelial cell(s) prior to the cell(s) being exposed to the small molecule; measuring a second level of SIRT1 expression after the cell(s) is exposed to the small molecule; and comparing the first level of SIRT1 expression with the second level of miRNA-204-5p expression.

In certain embodiments, the second level of SIRT1 expression is increased as compared to the first level of SIRT1 expression.

In certain embodiments, the method further comprises contacting the endothelial cell(s) with serum from an animal fed a high-fat diet prior to measuring the first level of miRNA-204-5p expression.

In certain embodiments, the level of miRNA-204-5p expression is measured using an activatable sensor oligonucleotide.

In certain embodiments, the miRNA-204-50 inhibitor is a small molecule identified using a method of any one of claims 20-28.

In certain embodiments, the present invention provides a method of treating endothelial dysfunction, cardiovascular disease and/or atherosclerosis in a mammal, comprising administering vector comprising an expression cassette comprising a promoter operably linked to a mircoRNA-204-5p inhibitor, to the mammal.

In certain embodiments, the vector is a plasmid.

In certain embodiments, the vector is a viral vector.

### BRIEF DESCRIPTION OF THE FIGURES

FIG. 1. Gut microbiota regulates miR-204, SIRT1 and vascular function. (A) Relative change (Δ) in expression of miR against log₂ expression of miRs (down-regulated miRs, up-regulated miRs, n=3). (B) A heat-map of selected miRs expression in GFM and PFM by RTqPCR and nCounter assay. (C) Potential target analysis (www.microRNA.org-miR-SVR score at threshold <−0.50, <−0.75 and <−0.90, covering ˜20000 (20K) mRNAs) of down-regulated miRs. SIRT1 falls in top 0.5% of potentially regulated target genes. (D & E) Change in TRPM3 and SIRT1 expression in aortas of GFM (shown as % of PFM). n=4, *P<0.05 vs. PFM. (F-J) Effect of antibiotic-induced gut microbiota suppression on aortic miR-204, TRPM3, SIRT1, and eNOS (shown as % of NPD). n=3-6. (K) Upper panel: miR-204 expression (purple chromogen) in mouse aortic vascular wall (counterstained with nuclear fast red, magnification ×100). Lower panel: SIRT1 (immunofluorescence) in mouse aortic vascular wall (counterstained with DAPI, magnification ×63). (L) Gut microbiota suppression improved endothelium-dependent vasorelaxation (NPD; filled circle, NPD-AB; open circle, NPD-AB-R; filled triangle). n=8-10 aortic rings from 3 mouse, *P<0.05 vs. NPD. #P<0.05 vs. NPD-AB. GFM; germ-free mice, PFM; pathogen-free mice, NPD; normal-pellet diet fed control mice, NPD-AB; NPD-fed mice receiving antibiotics, NPD-AB-R; NPD-AB mice after stoppage of antibiotics re-colonized with NPD mice. mSIRT1; SIRT1 mRNA, mTRPM3; TRPM3 mRNA. Error bar represents s.e.m.

FIG. 2. MiR-204 impairs endothelium-dependent vascular relaxation by downregulating SIRT1. (A) MiR-204 mimic (miR-204-M) decreases SIRT1 expression in human umbilical vein endothelial cells (HUVECs) as well as mouse aortic smooth muscle cells (MASMCs). n=3 independent experiments. (B) Impaired endothelium-dependent vascular-relaxation in aortic rings transfected ex-vivo with miR-204-M and adLacZ (open circle) compared to aortas transfected with scrambled miR and adLacZ (filled circle-). Adenovirus-mediated SIRT1 over-expression in miR-204-M transfected aortic rings ex vivo restores endothelium-dependent vascular relaxation (filled triangle). n=5-6 aortic rings from 3 mouse. adLacZ; control adenovirus expressing E. Coli LacZ, adSIRT1; adenovirus expressing SIRT1.* P<0.05 vs. scrambled control miR+adLacZ, # P<0.05 vs. miR-204-M+adLacZ. Error bar represents s.e.m.

FIG. 3. Atherogenic high-fat diet (HFD)-induced endothelial dysfunction is mediated through miR-204 and rescued with miR-204-inhibitor. (A-C) Effect of HFD feeding, gut-microbiota suppression by antibiotics and re-colonization on miR-204 (A and B) and level of SIRTlin aorta (C). Immunoblot-SIRT1 protein, Bar graph-SIRT1 mRNA. n=4-6. (D) HFD feeding impaired endothelium-dependent vascular relaxation (NPD; filled circle, HFD; open circle). Improved endothelium-dependent vascular relaxation in aortic rings isolated from HFD-AB (filled triangle) mice as compared to HFD, and was found to be reversed in aortic rings isolated from HFD-AB-R (open triangle). n=10-12 aortic rings. (E) HFD feeding impaired endothelium-dependent vascular relaxation in endothelium specific SIRT1 knockout (eSIRT1−/−) mouse (eSIRT1−/−NPD; filled circle, eSIRT1−/−HFD; open circle), and not rescued with the suppression of gut microbiota (filled triangle, eSIRT1−/−HFD-AB). n=6-12. *P<0.05 vs. eSIRT1−/−NPD. (F-H) Effect of systemic delivery of miR-204-inhibitor (mir-204-I) on aortic expression of miR-204 and SIRT1 in NPD and HFD-fed mouse. n=4-6. (I) Systemic delivery of miR-204-I improved endothelium-dependent vascular relaxation in aortic rings isolated from HFD-fed mouse as compared to HFD-fed mouse receiving scramble control (NPD-SC; filled circle, NPD-204-I; open triangle, HFD-SC; open circle, HFD-204-I; filled triangle). n=15-18 aortic rings. (J) Incubation of aortic rings with miR-204 inhibitor ex-vivo improved mSIRT1 level (error bar represents s.e.m of technical replicates). (K) Ex-vivo inhibition of aortic miR-204 in aortic rings isolated from HFD-fed mouse by miR-204-I improved endothelium-dependent vascular relaxation (NPD-SC; closed circle, HFD-SC; open circle, HFD-204-I; filled triangle). n=5-10 aortic rings. NPD; NPD-fed control mice, HFD; HFD-fed mice, HFD-AB; HFD-fed mice receiving antibiotics, HFD-AB-R; HFD-AB mice after stoppage of antibiotics re-colonized with HFD mice, eSIRT1−/−NPD; eSIRT1−/− mice on NPD, eSIRT1−/−HFD; eSIRT1−/− mice on HFD, eSIRT1−/−HFD-AB; eSIRT1−/− mice on HFD receiving antibiotics.*P<0.05 vs. NPD or NPD-SC. #P<0.05 vs. HFD or HFD-SC. mSIRT1; SIRT1 mRNA. Error bar represents s.e.m. (L) Effect of miR-204-I on the expression eNOS mRNA in isolated aorta from normal pellet diet (NPD) or atherogenic high-fat diet (HFD)-fed mouse ex-vivo. n=5-10 aortic rings from 2-3 mouse. *P<0.05 vs. NPD-SC. #P<0.05 vs. HFD-SC. Error bar represents s.e.m.

FIG. 4. (A) Heat-map of differentially regulated miRs in GFM and PFM (control). (B) Percent expression of most abundant miRs in aorta (mean of miRs from 3 mice). (C) Percent change in the expression of most abundant miRs in aortas of GFM compared to PFM. (D) Expression of dicer and drosha in aortas of GFM and PFM. n=3. (E) SIRT1 expression in aortas, skeletal muscle and lungs of GFM (% of PFM, n=4, *P<0.05). Error bar represents s.e.m.

FIG. 5. (A) Effect of antibiotics on mouse body weight. (B) Representative photographs of white adipose tissue (WAT) and brown adipose tissue (BAT) from NPD, NPD-AB and NPD-AB-R mice. (C) Effect of antibiotics on adiposity in mice. Adiposity was calculated by dividing the sum of WAT and BAT by body weight. (D) Effect of antibiotics on total cholesterol (open bar) and HDL-Cholesterol (Closed bar). NPD; normal-pellet diet fed control mice, NPD-AB; NPD-fed mice receiving antibiotics, NPD-AB-R; NPD-AB mice after stoppage of antibiotics re-colonization with NPD mice Error bar represents s.e.m.

FIG. 6. (A) Effect of miR-204 mimic transfection, and adenovirus encoding SIRT1 (adSIRT1) on expression of SIRT1 in endothelium of mouse aortas ex vivo. (B) MiR-204 expression in aortas transfected with miR-204 mimic and adSIRT1. SC: scrambled control miR; adLacZ: control adenovirus encoding E. Coli LacZ gene.

FIG. 7. Effect of long-term (16 weeks) HFD feeding and suppression of gut microbiota by antibiotics (total of 6 weeks, 10th to 16th week) on miR-204 (A) and SIRT1(B) expression in aorta. *P<0.05 vs. NPD. #P<0.05 vs. HFD. Error bar represents s.e.m.

FIG. 8. Representative photomicrographs showing SIRT1 immunostaining in aorta, lung liver and kidney of endothelial specific SIRT1 knockout (eSIRT1−/−) and wild type (WT) mouse (Magnification×40).

FIG. 9. (A) Decreased expression of miR-204 and increased expression of SIRT1 in HUVECs grown with media containing serum of mice kept on antibiotics containing water. n=3, representative of independent experiments. * P<0.05 vs. NPD. (B) Expression of miR-204 in HUVECs grown with media containing serum of either normal or recolonized mice. n=3. Error bar represents s.e.m.

FIG. 10. Effect of systemic delivery of miR-204-inhibitor (miR-204-I) on aortic expression of miR-204 (A) and SIRT1 (B) in NPD and HFD-fed mouse. n=4-6. (C) Improved endothelium-dependent vascular relaxation in aortic rings isolated from HFD-fed mice receiving miR-204-I as compared to HFD-fed mouse receiving scramble control (NPD-SC; filled circle, NPD-204-I; open triangle, HFD-SC; open circle, HFD-204-I; filled triangle). SC: Scramble control. n=15-18 aortic rings from 4-6 mouse. *P<0.05 vs. NPD-SC. #P<0.05 vs. HFD-SC. (D) Effect of serum isolated from normal pellet diet (NPD) or high-fat diet (HFD)-fed mouse on the miR-204-5p expression in human umbilical vein endothelial cells. *P<0.05 vs. NPD.

### DETAILED DESCRIPTION

Atherosclerosis is a vascular condition which initiates with the impairment in endothelial function, deposition of fat in the sub-endothelial layer and ultimately leading to the atheromatous plaque formation and obstruction in the blood flow in arteries. Impairment of endothelial function is a precursor to the atherosclerosis. The studies described herein reveal that microRNA-204-5p (miR-204-5p), a small non-coding RNA, impairs endothelial function by regulating expression of multiple genes, including SIRTUIN1 (SIRT1). Atherogenic high-fat diet promotes vascular expression of miR-204-5p and endothelial dysfunction, and blockade of the miR-204 function rescues atherogenic diet induced endothelial dysfunction in mice. Accordingly, certain embodiments of the invention provide methods of treating and/or preventing endothelial dysfunction, cardiovascular diseases and/or atherosclerosis by blocking the expression and/or activity of miR-204-5p. In certain embodiments, endothelial dysfunction, cardiovascular diseases and/or atherosclerosis may be treated and/or prevented by blocking miR-204 binding to its target genes using, e.g., inhibitors and/or sponges. In certain aspects of the invention, the methods described herein can offer prevention of the progression of disease with minimal change in life-style.

miRs

MicroRNAs (miRNAs) are small, non-coding RNA molecules which are able to regulate gene expression via two different mechanisms: a) translational inhibition or b) target mRNA cleavage. The choice of mechanism depends on the degree of complementarity between miRNA and the target gene in combination with a so-called Argonaute Protein. In the case of almost perfect complementarity, a cleavage of the target gene takes place with subsequent RNA degradation, whereas a translational inhibition takes place in the case of only partial complementarity. The total number of different miRNAs is estimated to be approximately 300-500; miRNAs thus constitute approximately 1% of the human genome. miRNAs have been discovered in various species and appear to be highly conserved. Although the target genes (or targets) and thus the biological functions of miRNAs have to date largely not been able to be identified, it is estimated that miRNAs regulate up to 30% of the genes of the human genome.

As described herein, miR-204-5p, a small non-coding RNA that targets SIRT1, has been shown to be upregulated in the vascular wall, especially the endothelium, and to promote endothelial dysfunction and atherosclerosis. This mechanism for the initiation and progression of atherosclerotic vascular disease is supported by data from an in vivo model of endothelial dysfunction (see, Examples). Human miR-204-5p may be referred to as hsa-miR-204-5p (Accession No.: MIMAT0000265; 5′-UUCCCUUUGUCAUCCUAUGCCU-3′ (SEQ ID NO:11)), but may also be referred to as hsa-miR-204, miR-204, or mmu-miR-204. The human form is compatible with both mouse (mmu-miR-204-5p) and rat (rno-miR-204-5p). The stem loop sequence for hsa-miR-204-5p (Accession No.: MI0000284) is: 5′-GGCUACAGUCUUUCUUCAUGUGACUCGUGGACUUCCCUUUGUCAUCCUAUGCCUGA GAAUAUAUGAAGGAGGCUGGGAAGGCAAAGGGACGUUCAAUUGUCAUCACUGGC-3′ (SEQ ID NO:23). The mature sequence for hsa-miR-204-3p is 3′-GCUGGGAAGGCAAAGGGACGU (Accession No. MIMAT0022693; SEQ ID NO: 27).

Hsa-miR-211-5p, which is also called hsa-miR-211 (Accession No.: MI0000287) is a small non-coding RNA that has the same seed sequence as that of miR-204. The sequence for hsa-miR-211-5p is: 5′-UCACCUGGCCAUGUGACUUGUGGGCUUCCCUUUGUCAUCCUUCGCCUAGGGCUCUGA GCAGGGCAGGGACAGCAAAGGGGUGCUCAGUUGUCACUUCCCACAGCACGGAG-3′ (SEQ ID NO: 28). The mature sequence for hsa-miR-211-5p is 5′-UUCCCUUUGUCAUCCUUCGCCU-3′ (SEQ ID NO: 29); and the mature sequence for hsa-miR-211-3p is 5′-GCAGGGACAGCAAAGGGGUGC-3′ (SEQ ID NO: 30).

Methods of Treatment

Certain embodiments of the invention provide a method of treating endothelial dysfunction, cardiovascular disease and/or atherosclerosis in a mammal (e.g., a human), comprising administering an effective amount of a miRNA-204-5p inhibitor to the mammal.

Certain embodiments of the invention provide a miRNA-204-5p inhibitor for use in treating endothelial dysfunction, cardiovascular disease and/or atherosclerosis in a mammal (e.g., a human).

Certain embodiments of the invention provide the use of a miRNA-204-5p inhibitor to prepare a medicament for treating endothelial dysfunction, cardiovascular disease and/or atherosclerosis in a mammal (e.g., a human).

Certain embodiments of the invention provide a miRNA-204-5p inhibitor for use in medical therapy.

As used herein, “treating” or “treatment” refers to reversing, alleviating, delaying the onset of, inhibiting the progress of, and/or preventing a disease or disorder, or one or more symptoms thereof, to which the term is applied in a subject. In some embodiments, treatment may be applied after one or more symptoms have developed. In other embodiments, treatment may be administered in the absence of symptoms. For example, treatment may be administered prior to symptoms (e.g., in light of a history of symptoms and/or one or more other susceptibility factors), or after symptoms have resolved, for example to prevent or delay their reoccurrence.

As used herein, a miRNA-204-5p inhibitor refers to a molecule capable of reducing the expression and/or function of miRNA-204-5p in a cell. In certain embodiments, the miRNA-204-5p inhibitor reduces miRNA-204-5p expression. In certain embodiments, the miRNA-204-5p inhibitor reduces miRNA-204-5p function. In certain embodiments, the expression and/or function of miRNA-204-5p in a cell is reduced by about 1, 5, 10, 15, 20, 25, 30, 35, 40, 45, 50, 55, 60, 65, 70, 75, 80, 85, 90, 95, or 100%, as compared to the expression or function of a miRNA-204-5p in a cell (e.g., in a mammal) that has not been administered/contacted with the inhibitor. miRNA-204-5p expression and/or function may be determined using assays known in the art. For example, miRNA expression may be measured using an activatable sensor oligonucleotide, RT-PCR, Real-Time qRT-PCR, or northern blotting, or in-situ hybridization, whereas miRNA function may be measured by examining the expression of miRNA targets.

Inhibitors may interact directly or indirectly with miR-204-5p and include, for example, oligonucleotides, including miRNA sponges (Ebert et al., Nature Methods, 4, 721-726 (2007)), and small molecules.

In certain embodiments, the administration of the micro-RNA-204-5p inhibitor causes increased SIRTUIN1 (SIRT1) expression in a cell in the mammal, as compared to SIRT1 expression in a cell in a mammal that was not administered the micro-RNA-204-5p inhibitor. In certain embodiments, SIRT1 expression is increased by about 1, 5, 10, 15, 20, 25, 30, 35, 40, 45, 50, 55, 60, 65, 70, 75, 80, 85, 90, 95, or 100%, as compared to SIRT1 expression in a cell in a mammal that was not administered the micro-RNA-204-5p inhibitor. In certain embodiments, SIRT1 expression is increased in aortic endothelial cells.

In certain embodiments, the administration of the micro-RNA-204-5p inhibitor causes increased endothelial nitric oxide synthase (eNOS) expression in a cell in the mammal, as compared to eNOS expression in a cell in a mammal that was not administered the micro-RNA-204-5p inhibitor. In certain embodiments, eNOS expression is increased by about 1, 5, 10, 15, 20, 25, 30, 35, 40, 45, 50, 55, 60, 65, 70, 75, 80, 85, 90, 95, or 100%, as compared to eNOS expression in a cell in a mammal that was not administered the micro-RNA-204-5p inhibitor. In certain embodiments, eNOS expression is increased in aortic endothelial cells.

As used herein, SIRT1/eNOS expression may be used to refer to expression of the RNA transcript or the translated protein, Expression may be determined using assays known in the art, for example, RT-PCR, Real-Time qRT-PCR, Western blot, immunofluorescence, or immunocytochemistry.

In certain embodiments, the miRNA-204-5p inhibitor comprises an oligonucleotide (e.g., a deoxyribooligonucleotide or a ribooligonucleotide) comprising 4 to 7 nucleotides (e.g., 4, 5, 6 or 7 nucleotides) that are complementary to a seed sequence of miRNA-204-5p (UCCCUUU SEQ ID NO:24. Thus, the sequence of the inhibitor comprises 4 to 7 nucleotides (e.g., 4, 5, 6 or 7 nucleotides) of the sequence AGGGAAA (SEQ ID NO: 26).

In certain embodiments, the miRNA-204-5p inhibitor comprises an oligonucleotide (e.g., a deoxyribooligonucleotide or a ribooligonucleotide) comprising a sequence having at least about 70% complementarity with a seed sequence of miRNA-204-5p (5′-UCCCUUU-3′ SEQ ID NO:24). In certain embodiments, the oligonucleotide comprises a sequence having at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% complementarity to a seed sequence of miRNA-204-5p.

In certain embodiments, the miRNA-204-5p inhibitor comprises an oligonucleotide (e.g., a deoxyribooligonucleotide or a ribooligonucleotide) comprising a sequence having at least about 70% complementarity with SEQ ID NO:11 (i.e., miRNA-204-5p). In certain embodiments, the oligonucleotide comprises a sequence having at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% complementarity with SEQ ID NO:11.

In certain embodiments, the miRNA-204-5p inhibitor comprises an oligonucleotide comprising a sequence having at least about 70% sequence identity to SEQ ID NO:20 (i.e., miR-204-I). In certain embodiments, the oligonucleotide comprises a sequence having at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:20. In certain embodiments, the oligonucleotide consists of SEQ ID NO:20.

In certain embodiments, the miRNA-204-5p inhibitor comprises an oligonucleotide (e.g., an antisense oligonucleotide; e.g., a deoxyribooligonucleotide or a ribooligonucleotide) comprising a sequence having at least about 70% complementarity with miRNA-211 (Accession No. MIMAT0000268; 5′-UUCCCUUUGUCAUCCUUCGCCU-3′ SEQ ID NO:25). In certain embodiments, the oligonucleotide comprises a sequence having at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% complementarity with miR-211.

In certain embodiments, the oligonucleotide is 15 to 60 nucleotides in length]. In certain embodiments, the oligonucleotide is 15-50, 15-45, 15-40, 15-35, 15-30 or 15-25 nucleotides in length or 18, 19, 20, 21, 22, 23, 24 or 25 nucleotides in length.

The present invention provides modified and unmodified nucleic acids (e.g., oligonucleotides). Accordingly, in certain embodiments, an oligonucleotide may contain one or more chemical modifications to the nucleotides themselves (e.g., locked nucleic acids), the nucleoside linkage (e.g., phosphorothioate, methyl phosphonate, phosphoramidate, or a combination thereof), or to the sugar moiety.

In certain embodiments, the miRNA-204-5p inhibitor comprises a miRNA sponge. As used herein a “miRNA sponge” refers to a RNA transcript containing multiple, tandem binding sites to a microRNA of interest (e.g., miRNA-204-5p); sponges specifically inhibit microRNAs with a complementary heptameric seed, such that a single sponge can be used to block an entire microRNA seed family (Ebert et al., Nature Methods, 4, 721-726 (2007)). Sponges generally consist of multiple binding sites to the miRs (ranging from, but not limited to, 3-10 nucleotides). Another way to inhibit miR function involves using TuD (Tough Decoys) RNAs. TuDs have miRs binding site (s) in the single stranded regions of short stem-loops. In certain embodiments, an oligonucleotide or miRNA sponge described herein is chemically synthesized. In certain embodiments, an oligonucleotide or miRNA sponge described herein is expressed in vivo from a vector. Accordingly, certain embodiments of the invention provide a method of treating endothelial dysfunction, cardiovascular disease and/or atherosclerosis in a mammal, comprising administering a vector comprising an expression cassette comprising a promoter operably linked to a mircoRNA-204-5p inhibitor (e.g., an oligonucleotide or miRNA sponge as described herein), to the mammal. In certain embodiments, the vector is a plasmid. In certain embodiments, vector is a viral vector.

In certain embodiments, the miRNA-204-5p inhibitor comprises a small molecule inhibitor. In certain embodiments the small molecule inhibits binding between miRNA-204-5p and a target gene (e.g., SIRT1 and/or eNOS). In certain embodiments, the small molecule inhibitor is an inhibitor identified using a method described herein.

Certain embodiments of the invention provide a method of decreasing dysfunction in an endothelial cell that expresses micro-RNA-204-5p, comprising contacting the cell in vivo or in vitro with a micro-RNA-204-5p inhibitor.

Certain embodiments of the invention provide a method of increasing SIRT1 and/or eNOS expression in an endothelial cell that expresses micro-RNA-204-5p and SIRT1 and/or eNOS, comprising contacting the cell in vivo or in vitro with a micro-RNA-204-5p inhibitor. In certain embodiments, the endothelial cell is an aortic endothelial cell.

Methods of Identifying Small Molecule Inhibitors of miRNA-204-5p

Certain embodiments of the invention provide methods of identifying compounds (e.g., small molecules) that can be used to inhibit the expression and/or function of miRNA-204-5p, and thus serve as pharmaceuticals to treat endothelial dysfunction, cardiovascular disease and/or prevent atherosclerosis. As described herein, miRNA-204-5p downregulates SIRT1 expression. Additionally, the serum from mice, when fed a high-fat diet, has also been shown to downregulate endothelial SIRT1 expression (see, Example 1). Accordingly, SIRT1 expression may also be used to identify small molecule miRNA-204-5p inhibitors.

Thus, certain embodiments of the invention provide a method of identifying a miRNA-204-5p inhibitor comprising comparing the level of miRNA-204-5p expression in an endothelial cell before and after exposure to a small molecule, wherein the small molecule is a miRNA-204-5p inhibitor if the level of miRNA-204-5p expression is decreased in the endothelial cell after exposure to the small molecule. In certain embodiments, the methods further comprise comparing the level of SIRT1 expression in the endothelial cell before and after exposure to the small molecule. As discussed herein, miRNA-204-5p downregulates SIRT1. Therefore, if the small molecule is a miRNA-204-5p inhibitor, the level of SIRT1 expression may be increased in the endothelial cell exposed to the small molecule. Accordingly, in certain embodiments, the level of SIRT1 expression is increased in the endothelial cell after exposure to the small molecule. In certain embodiments, the endothelial cell(s) is exposed to serum from an animal (e.g., a mouse) fed a high fat diet prior to exposure to the small molecule (e.g., see, the Examples).

Certain embodiments of the invention provide a method of identifying a miRNA-204-5p inhibitor comprising measuring a first level of miRNA-204-5p expression in an endothelial cell(s); contacting the endothelial cell(s) with a small molecule; measuring a second level of miRNA-204-5p expression in the endothelial cell(s); and comparing the first level of miRNA-204-5p expression with the second level of miRNA-204-5p expression, wherein, the small molecule is a miRNA-204-5p inhibitor if the second level of expression is decreased as compared to the first level of expression. In certain embodiments, the methods further comprise measuring a first level of SIRT1 expression in the endothelial cell(s) prior to the cell(s) being exposed to the small molecule; measuring a second level of SIRT1 expression after the cell(s) is exposed to the small molecule; and comparing the first level of SIRT1 expression with the second level of miRNA-204-5p expression. In certain embodiments, the second level of SIRT1 expression is increased as compared to the first level of SIRT1 expression. In certain embodiments, the method further comprises contacting the endothelial cell(s) with serum from an animal (e.g., a mouse) fed a high-fat diet prior to measuring the first level of miRNA-204-5p/SIRT1 expression.

In certain embodiments, miR-204-5p expression may be measured using an activatable sensor oligonucleotide, which is cleavable, typically completely complementary to a target miRNA, and dual-labeled with a fluorescent dye and a quencher (Yoo et al., Detection of miRNA expression in intact cells using activatable sensor nucleotides, Chemistry & Biology, 2014, 21, 199-204). Upon entering a cell, the sensor oligonucleotide binds its specific miRNA target, which triggers assembly of RISC around the miRNA-sensor duplex, the subsequent cleavage of the sensor oligonucleotide, and fluorescence, which may then be measured.

In certain other embodiments, miRNA-204-5p or SIRT1 expression may be measured using RT-PCR, Real-Time qRT-PCR, northern blotting, or in-situ hybridization.

In certain other embodiments, SIRT1 expression may be measured using a SIRT1 promoter-reporter assay.

In certain embodiments, SIRT1 expression may be measured at the protein level using Western Blot, Immunofluorescence, immunocytochemistry, or immunohistochemistry.

General Terminology and Methods

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. An “allele” is one of several alternative forms of a gene occupying a given locus on a chromosome.

The term “endogenous gene” refers to a native gene in its natural location in the genome of an organism.

A “transgene” refers to a gene that has been introduced into the genome by transformation. Transgenes include, for example, DNA that is either heterologous or homologous to the DNA of a particular cell to be transformed. Additionally, transgenes may include native genes inserted into a non-native organism, or chimeric genes.

The term “nucleic acid” refers to deoxyribonucleic acid (DNA) or ribonucleic acid (RNA) and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

A “nucleotide sequence” is a polymer of DNA or RNA that can be single-stranded or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. The RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

A “variant” of a molecule is a sequence that is substantially similar to the sequence of the native molecule. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

As used herein, the term “recombinant nucleic acid”, e.g., “recombinant DNA/RNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome which has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from a source by chemical means, e.g., by the use of restriction endonucleases, so that it can be further manipulated, e.g., amplified, for use in the invention, by the methodology of genetic engineering. “Recombinant DNA” includes completely synthetic DNA sequences, semi-synthetic DNA sequences, DNA sequences isolated from biological sources, and DNA sequences derived from RNA, as well as mixtures thereof.

The term “RNA transcript” or “transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell.

The following terms are used to describe the sequence relationships between two or more nucleic acids or polynucleotides: (a) “reference sequence,” (b) “comparison window,” (c) “sequence identity,” (d) “percentage of sequence identity,” (e) “substantial identity”, (1) “complementary”, (g) “percent complementarity” and (h) “substantial complementarity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity or percent complementarity between any two sequences can be accomplished using a mathematical algorithm.

Computer implementations of these mathematical algorithms can be utilized for comparison of sequences to determine sequence identity, including complementarity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g. BLASTN for nucleotide sequences) can be used. The BLASTN program (for nucleotide sequences) uses as defaults a wordlength (W) of 11, an expectation (E) of 10, a cutoff of 100, M=5, N=−4, and a comparison of both strands. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

(c) As used herein, “sequence identity” or “identity” in the context of two nucleic acid sequences makes reference to a specified percentage of nucleotides in the two sequences that are the same when aligned for maximum correspondence over a specified comparison window, as measured by sequence comparison algorithms or by visual inspection.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters.

(f) “Complementary” as used herein refers to the broad concept of subunit sequence complementarity between two nucleic acids, e.g., an antisense oligonucleotide and a miRNA. When a nucleotide position in both of the molecules is occupied by nucleotides normally capable of base pairing with each other, then the nucleic acids are considered to be complementary to each other at this position.

(g) As used herein, “percent complementarity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of corresponding positions in each of the molecules that are occupied by nucleotides which normally base pair with each other (e.g., A:T and G:C nucleotide pairs), dividing the number of positions by the total number of positions in the window of comparison, and multiplying the result by 100 to yield the percentage of complementarity.

(h) Two nucleic acids are “substantially complementary” to each other when at least about 50%, at least about 60%, at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of corresponding positions in each of the nucleic acid molecules are occupied by nucleotides which normally base pair with each other (e.g., A:T and G:C nucleotide pairs).

Another indication that nucleotide sequences are substantially identical/complementary is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tm is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tm can be approximated from the equation: Tm 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tm is reduced by about 1° C. for each 1% of mismatching; thus, Tm, hybridization, and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tm) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook and Russell 2001, for a description of SSC buffer). Often, a high stringency wash is preceded by a low stringency wash to remove background probe signal. For short nucleic acid sequences (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. Stringent conditions may also be achieved with the addition of destabilizing agents such as formamide. In general, a signal to noise ratio of 2× (or higher) than that observed for an unrelated probe in the particular hybridization assay indicates detection of a specific hybridization. Very stringent conditions are selected to be equal to the Tm for a particular nucleic acid molecule.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

General methods for constructing recombinant DNA that can be introduced into target cells are well known to those skilled in the art, and the same compositions and methods of construction may be utilized to produce the DNA useful herein.

A “vector” is defined to include, inter alia, any viral vector, as well as any plasmid, cosmid, phage or binary vector in double or single stranded linear or circular form that may or may not be self-transmissible or mobilizable, and that can transform prokaryotic or eukaryotic host either by integration into the cellular genome or exist extrachromosomally (e.g., autonomous replicating plasmid with an origin of replication).

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. The coding region usually codes for a functional RNA of interest, for example an miRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like. Reporter genes that encode for easily assayable proteins are also well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

“Regulatory sequences” are nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted herein, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. Examples of promoters that may be used in the present invention include the mouse U6 RNA promoters, synthetic human H1RNA promoters, SV40, CMV, RSV, RNA polymerase II and RNA polymerase III promoters.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of antisense oligonucleotides or miRNAs, expression may refer to the transcription of the oligonucleotide or miRNA only. In addition, expression refers to the transcription and stable accumulation of sense (mRNA) or functional RNA. Expression may also refer to the production of protein.

“Altered levels” refers to the level of expression in transgenic cells or organisms that differs from that of normal or untransformed cells or organisms.

“Overexpression” refers to the level of expression in transgenic cells or organisms that exceeds levels of expression in normal or untransformed cells or organisms.

“Antisense inhibition” refers to antisense RNA transcripts capable of suppressing the expression of miRNA or protein from an endogenous gene or a transgene.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

The terms “cis-acting sequence” and “cis-acting element” refer to DNA or RNA sequences whose functions require them to be on the same molecule. An example of a cis-acting sequence on the replicon is the viral replication origin.

The terms “trans-acting sequence” and “trans-acting element” refer to DNA or RNA sequences whose function does not require them to be on the same molecule.

“Chromosomally-integrated” refers to the integration of a foreign gene or nucleic acid construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

“Transformed,” “transduced,” “transgenic” and “recombinant” refer to a host cell into which a heterologous nucleic acid molecule has been introduced. As used herein the term “transfection” refers to the delivery of DNA into eukaryotic (e.g., mammalian) cells. The term “transformation” is used herein to refer to delivery of DNA into prokaryotic (e.g., E. coli) cells. The term “transduction” is used herein to refer to infecting cells with viral particles. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

A “host cell” is a cell that has been transformed/transfected, or is capable of transformation/transfection, by an exogenous nucleic acid molecule. Host cells containing the transformed/transfected nucleic acid fragments are referred to as “transgenic” cells.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

Recombinant DNA can be readily introduced into the host cells, e.g., mammalian, bacterial, yeast or insect cells via an expression vector by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described herein below, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like. See, for example, U.S. Pat. Nos. 5,350,674 and 5,585,362. Thus, as will be apparent to one of ordinary skill in the art, a variety of suitable viral expression vectors are available for transferring exogenous nucleic acid material into cells. The selection of an appropriate expression vector to express a therapeutic agent for a particular condition amenable to gene silencing therapy and the optimization of the conditions for insertion of the selected expression vector into the cell, are within the scope of one of ordinary skill in the art without the need for undue experimentation.

In another embodiment, an expression vector may be in the form of a plasmid, which is transferred into the target cells by one of a variety of methods: physical (e.g., microinjection, electroporation, scrape loading, microparticle bombardment) or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (Promega®, Madison, Wis.). However, the efficiency of transfection by these methods is highly dependent on the nature of the target cell and accordingly, the conditions for optimal transfection of nucleic acids into cells using the herein-mentioned procedures must be optimized. Such optimization is within the scope of one of ordinary skill in the art without the need for undue experimentation.

In one embodiment, cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent (e.g., a miRNA-204-5p inhibitor) and the therapeutic agent is delivered in situ, for example, injecting the vector into the recipient.

As used herein, “exogenous nucleic acid material” refers to a nucleic acid or an oligonucleotide, either natural or synthetic, which is not naturally found in the cells; or if it is naturally found in the cells, is modified from its original or native form. Thus, “exogenous nucleic acid material” includes, for example, a non-naturally occurring nucleic acid that can be transcribed into an anti-sense RNA, as well as a “heterologous gene” (i.e., a gene encoding a protein that is not expressed or is expressed at biologically insignificant levels in a naturally-occurring cell of the same type). To illustrate, a synthetic or natural gene encoding human erythropoietin (EPO) would be considered “exogenous nucleic acid material” with respect to human peritoneal mesothelial cells since the latter cells do not naturally express EPO. Still another example of “exogenous nucleic acid material” is the introduction of only part of a gene to create a recombinant gene, such as combining a regulatable promoter with an endogenous coding sequence via homologous recombination.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the preselected DNA segment is being expressed. Expression may be evaluated by specifically identifying the RNA or peptide products of the introduced recombinant DNA sequences or evaluating the phenotypic changes brought about by the expression of the introduced recombinant DNA segment in the host cell.

The terms “protein,” “peptide” and “polypeptide” are used interchangeably herein.

Administration

The miRNA-204-5p inhibitors described herein can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the inhibitors may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the inhibitors to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the inhibitors can be determined by comparing their in vitro activity, and in vivo activity in animal models. Methods for the extrapolation of effective dosages in mice, and other animals, to humans are known to the art; for example, see U.S. Pat. No. 4,938,949.

The amount of the inhibitor, or an active salt or derivative thereof, required for use in treatment will vary not only with the particular salt selected but also with the route of administration, the nature of the condition being treated and the age and condition of the patient and will be ultimately at the discretion of the attendant physician or clinician.

The inhibitor may be conveniently formulated in unit dosage form; in one embodiment, the invention provides a composition comprising a miRNA-204-5p inhibitor formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

In certain embodiments, the miRNA-204-5p inhibitor is an oligonucleotide as described herein (e.g., an antisense oligonucleotide). In certain embodiments, the oligonucleotide is administered via intravenous injection. In certain embodiments, the oligonucleotide is administered weekly, e.g., for 2, 3, 4, 5, 6, 7, 8, 9 or more weeks. As discussed above, useful dosages of the inhibitors depend on a number of factors and may be determined by one skilled in the art. However, in certain embodiments, a suitable dose will be in the range of from about 0.5 to about 100 mg/kg, e.g., from about 1 to about 75 mg/kg of body weight, such as 1 to about 50 mg per kilogram body weight of the recipient, 1 to 10 mg/kg, or 1, 2, 3, 4, 5, 6, 7, 8, 9 or 10 mg/kg administered weekly. In certain embodiments, the oligonucleotide may be administered in combination with liposomes/microvesicles/cholesterol, which may improve delivery, stability and/or distribution of the oligonucleotide, and thereby its therapeutic benefits. Additionally, as discussed herein, an oligonucleotide described herein may be chemically modified, which may improve its delivery, stability and/or distribution, and thereby its therapeutic benefits. Accordingly, in certain embodiments, the oligonucleotide may be chemically modified, e.g., the backbone may be chemically modified.

The inhibitors can also be administered in combination with other therapeutic agents, for example, other agents that are useful for the treatment of cardiovascular disease, endothelial dysfunction and/or atherosclerosis. Examples of such agents include Cholesterol lowering therapeutic agents (e.g., atorvastatin, pravastatin), anti-hypertensive agents (e.g., propranolol, atenolol, enalapril), and/or dietary supplements (e.g. folic acids). Accordingly, in one embodiment the invention also provides a composition comprising a miRNA-204-5p inhibitor, or a pharmaceutically acceptable salt thereof, at least one other therapeutic agent, and a pharmaceutically acceptable diluent or carrier. The invention also provides a kit comprising a miRNA-204-5p inhibitor, or a pharmaceutically acceptable salt thereof, at least one other therapeutic agent, packaging material, and instructions for administering the miRNA-204-5p inhibitor or the pharmaceutically acceptable salt thereof and the other therapeutic agent or agents to a mammal to treat of cardiovascular disease, endothelial dysfunction and/or atherosclerosis.

The invention will now be illustrated by the following non-limiting Example.

### Example 1

### The Gut Flora Promotes Endothelial Dysfunction Through Vascular microRNA-204-Mediated Down-Regulation of Endothelial SIRTUIN1

Abstract

The gut flora contributes to development of atherosclerosis. Endothelial dysfunction, one manifestation of which is impaired endothelium-dependent vasorelaxation, accompanies and promotes atherosclerotic vascular disease. Here we show that gut flora impair endothelium-dependent vasorelaxation by remotely up-regulating microRNA-204 (miR-204) which downregulates SIRTUIN1 (SIRT1) in the vascular wall. Microarray analysis in aortas of germ-free mice revealed a set of down-regulated microRNAs, including miR-204, which target SIRT1. Suppression of gut flora in mice with antibiotics in drinking water decreased aortic miR-204, increased aortic SIRT1, and improved endothelium-dependent vasorelaxation, effects that were reversed with discontinuation of antibiotics. In addition, miR-204 mimic impaired endothelium-dependent aortic vasorelaxation ex vivo. Moreover, high-fat diet feeding stimulated aortic miR-204, suppressed SIRT1, and impaired endothelial function, all of which were mitigated by administration of antibiotics, and reversed with stoppage of antibiotics. In contrast, antibiotics did not improve high-fat diet-induced endothelial dysfunction in mice conditionally lacking endothelial SIRT1. In addition, anti-miR-204 delivered systemically prevented high-fat diet-induced endothelial dysfunction and vascular SIRT1 decrease. Finally, serum from mice on antibiotics suppressed miR-204, and increased SIRT1, in endothelial cells, effects that were not observed with serum from mice in which antibiotics were discontinued. Therefore, the gut flora remotely downregulates endothelial SIRT1 through miR-204, leading to impairment of endothelial function.

Introduction

The gut flora, consisting of trillions of organisms, plays an essential role in dietary energy harvest, and has been implicated in the pathogenesis of metabolic disorders such as diabetes (Qin, J. et al., Nature 490, 55-60, (2012), obesity (Backhed, F. et al., Proceedings of the National Academy of Sciences of the United States of America 101, 15718-15723, (2004)) as well as atherosclerotic vascular disease (Wang, Z. et al., Nature 472, 57-63, (2011); Bennett, B. J. et al., Cell metabolism 17, 49-60, (2013); Koeth, R. A. et al., Nature medicine 19, 576-585, (2013); Tang, W. H. et al., The New England journal of medicine 368, 1575-1584, (2013)). Atherosclerosis promoted by intestinal microbiota is attributed, in part, to augmented cholesterol accumulation in macrophages leading to foam cell formation (Wang, Z. et al., Nature 472, 57-63, (2011)). Impaired endothelial function is a precursor and strong predictor of atherosclerosis (Davignon, J. & Ganz, P., Circulation 109, 11127-32, (2004)). Both circulating pro-atherosclerotic phospholipid metabolites (Wang, Z. et al., Nature 472, 57-63, (2011); Koeth, R. A. et al., Nature medicine 19, 576-585, (2013)) and macrophage microRNAs (miRs) (Wang, D. et al., Circulation research 111, 967-981, (2012)) have been linked to accelerated foam cell formation induced by the gut flora, however, it is unclear whether the gut flora impacts vascular disease process by remotely regulating microRNA expression in the vascular wall.

Results

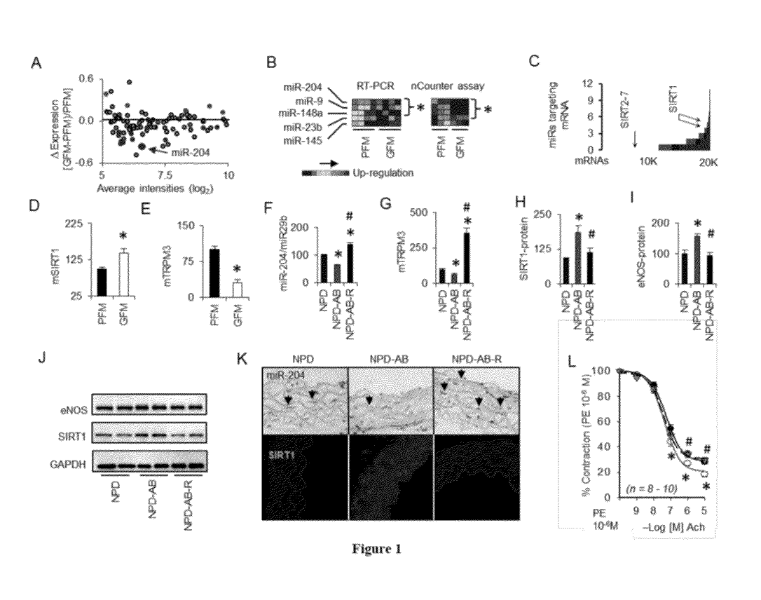

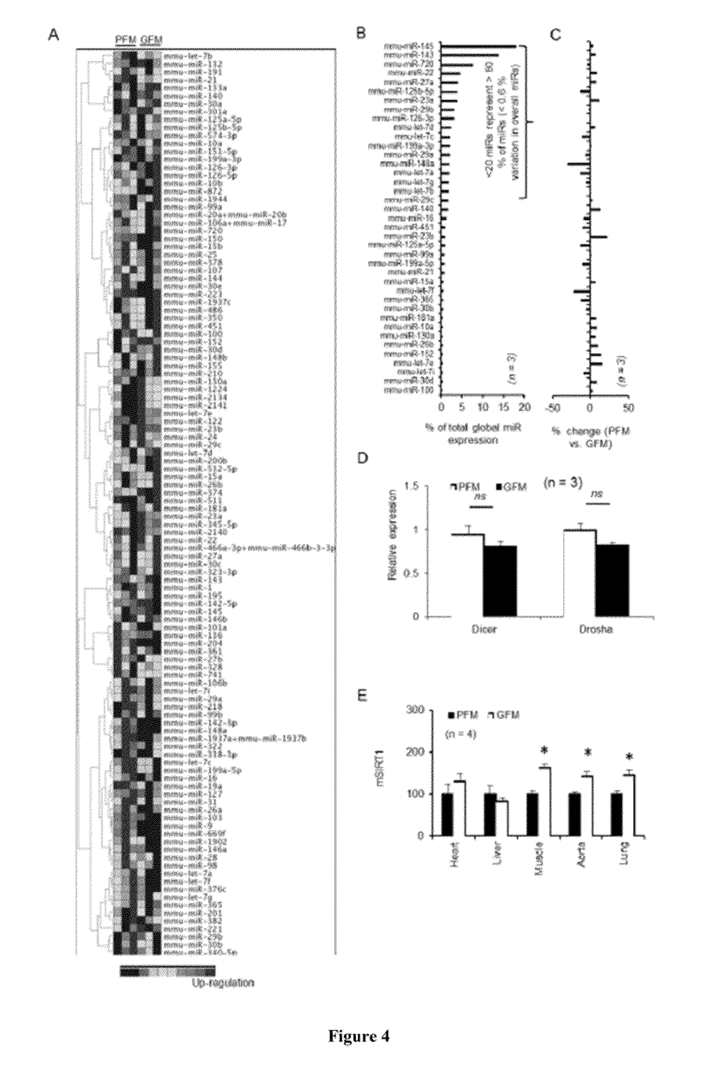

As described herein, the effect of the intestinal microbiome on vascular microRNA expression profiles was investigated. Unbiased microRNA nCounter expression arrays were performed and compared in aortas of germ-free mice (GFM) and pathogen free mice (PFM). Of the 578 microRNAs assayed, fifteen miRs were found to be significantly down-regulated while five miRs were up-regulated in GFM compared to PFM (FIG. 1A, FIG. 4A). Global miRNA expression and expression of the most abundant vascular miRNAs did not differ between GFM and PFM (FIGS. 4B& C). Similarly, expression of miRNA processing enzymes Dicer and Drosha were not altered in germ-free conditions (FIG. 4D). The changes in expression of miRNAs detected by nCounter array were validated by RTqPCR, confirming down-regulation of miR-204, miR-9 and miR-148a, and up-regulation of miR-23b in aortas of GFM (FIG. 1B).

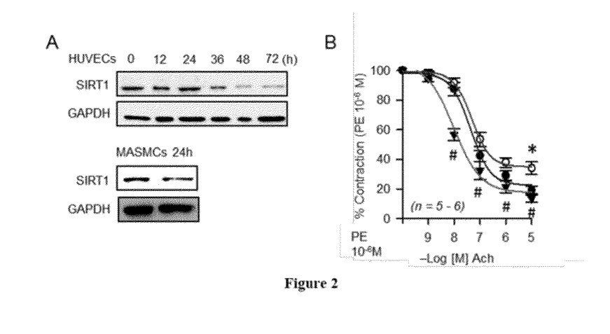

Computational mRNA target prediction showed SIRT1 to be in the top 100 genes (0.5 percentile of all genes) targeted by the conserved microRNAs identified to be down-regulated by nCounter array in aortas of GFM (FIG. 1C), among which miR-204, miR-9, miR-142-3p, and miR-199a-5p have been experimentally shown to down-regulate SIRT1. SIRT1 is a class III histone deacetylase expressed in cell types comprising the vascular wall and its expression is governed by microRNAs (Imai, S., et al. Nature 403, 795-800, (2000); Mattagajasingh, I. et al., Proceedings of the National Academy of Sciences of the United States of America 104, 14855-14860, (2007); Zhang, Q. J. et al., Cardiovascular research 80, 191-199, (2008); Choi, S. E. & Kemper, J. K., Molecules and cells 36, 385-392, (2013)). Since miR-204 was the most robustly down-regulated microRNA, this microRNA was further examined. It was confirmed that SIRT1 is a target of miR-204 in vascular endothelial cells and smooth muscle cells using a miR-204 mimic (miR-204-M) (FIG. 2A). Furthermore, SIRT1 mRNA was upregulated in several mouse tissues, including aortas (FIG. 1D), skeletal muscle and lungs, but not in heart and liver of GFM (FIG. 4E). Because miR-204 is in intron 6 of the TRPM3 gene located on chromosome 9q21.12, and is co-expressed with TRPM3 (Karali, M., et al., Investigative ophthalmology & visual science 48, 509-515, (2007)), TRPM3 expression in PFM and GFM was also compared. TRPM3 was down-regulated in GFM aortas (FIG. 1E).

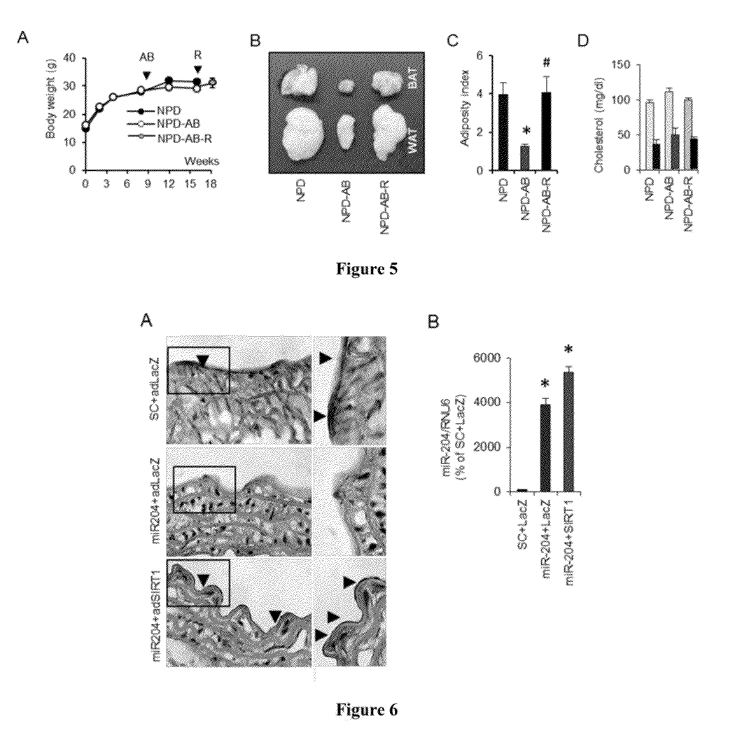

Whether suppressing gut flora biomass with broad spectrum antibiotics results in changes in miR-204 and SIRT1 expression similar to that observed in GFM was also examined. Mice removed from the larger colony and treated with antibiotics in drinking water for 6 weeks were leaner than their untreated counterparts (FIG. 5A-C), displayed lower miR-204 and TRPM3 (FIGS. 1F, G & K), and higher SIRT1 (FIGS. 1H, J & K), in their aortas. Antibiotic treatment also upregulated endothelial nitric oxide synthase (eNOS) (FIGS. 1J & K) and improved endothelium-dependent vasorelaxation (FIG. 1L). Withdrawal of antibiotics and co-habitation with mice not on antibiotics reversed changes in aortic expression of miR-204, TRPM3, SIRT1 and eNOS, and decreased endothelium-dependent vasorelaxation (FIG. 1F-L).

To establish a causal role for miR-204 in endothelial dysfunction, miR-204 mimic (miR-204-M) was transfected into mouse aortas ex vivo. MiR-204-M decreased endothelial SIRT1 (FIG. 6A), and impaired endothelium-dependent vasorelaxation (FIG. 2B). Moreover, adenoviral-mediated delivery of SIRT1 lacking the 3′-UTR, restored aortic SIRT1 expression suppressed by miR-204-M (FIG. 6B), and rescued endothelial dysfunction triggered by miR-204-M (FIG. 2B).

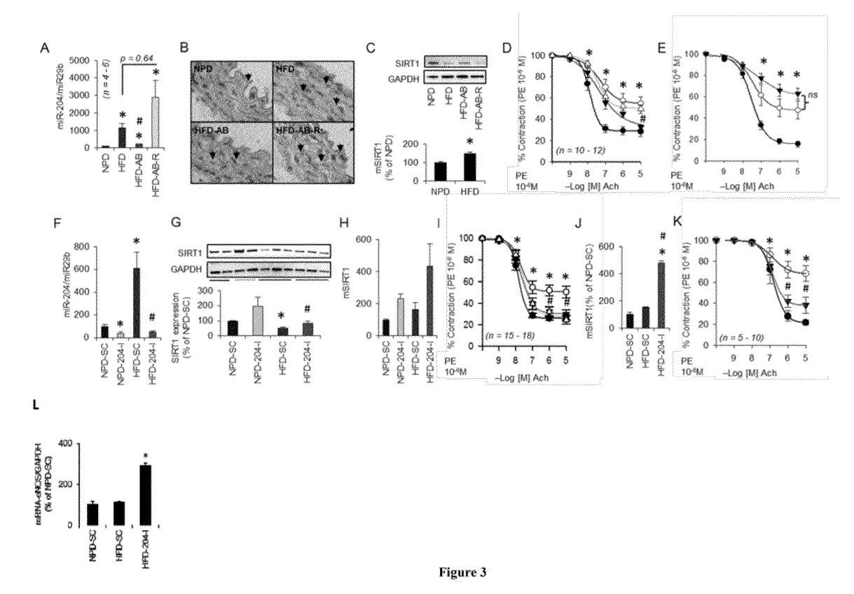

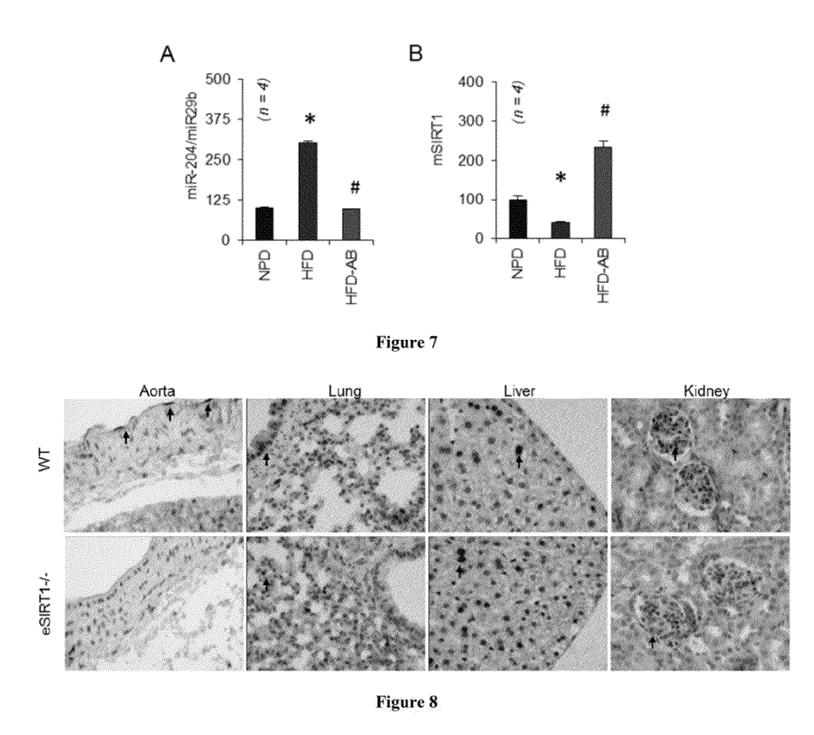

Whether aortic expression of SIRT1 and miR-204 are dependent on the gut flora in a high-fat diet (HFD) feeding model of endothelial dysfunction and atherosclerosis was also examined (Davis, N., Katz, S. & Wylie-Rosett, J., Cardiology in review 15, 62-66, (2007)). HFD feeding for 8 weeks increased miR-204 (FIGS. 3A & B), decreased SIRT1 protein (FIG. 3C, top) in mouse aortas, and impaired endothelium-dependent vasorelaxation (FIG. 3D). Although, aortic SIRT1 mRNA increased after 8 weeks of HFD (FIG. 3C, bottom), a longer course of HFD resulted in decrease in aortic SIRT1 mRNA (FIG. 7), suggesting a compensatory acute response in SIRT1 mRNA to decrease in protein. Broad-spectrum antibiotics delivered during the last six weeks of the HFD feeding period decreased vascular miR-204 (FIGS. 3A & B), restored SIRT1 (FIGS. 3C & 7), and rescued endothelial function (FIG. 3D). Removal of antibiotics and co-housing with mice not on antibiotics for two weeks reversed antibiotic-induced changes in aortic miR-204 and SIRT1 (FIG. 3A-C & 7), and negated the improvement in endothelium-dependent vasorelaxation observed with antibiotics (FIG. 3D). The role of vascular endothelial SIRT1 in the salutary effect of antibiotics on vasomotor function was also assessed using mice with conditional deletion of endothelial SIRT1 (eSIRT1−/−) (FIG. 8). Impaired endothelium-dependent vascular relaxation in response to HFD was not rescued by antibiotics in eSIRT1−/− (FIG. 3E), underscoring the vital role for endothelial SIRT1 in mediating the effect of antibiotics on vascular function.

Whether vascular miR-204 is responsible for endothelial dysfunction with HFD feeding was also investigated. HFD-stimulated aortic miR-204 was blunted by delivering locked nucleic acid anti-miR-204 with osmotic mini-pumps for six weeks of the feeding period. Blocking aortic miR-204 upregulation induced by HFD with anti-miR-204 (FIG. 3F) prevented endothelial dysfunction (FIG. 31) and rescued aortic SIRT1 down-regulation (FIG. 3G). Moreover, ex-vivo inhibition of miR-204 with anti-miR-204 in aortas of mice subjected to HFD feeding, increased vascular expression of SIRT1 (FIG. 3J) and mitigated endothelial dysfunction (FIG. 3K).

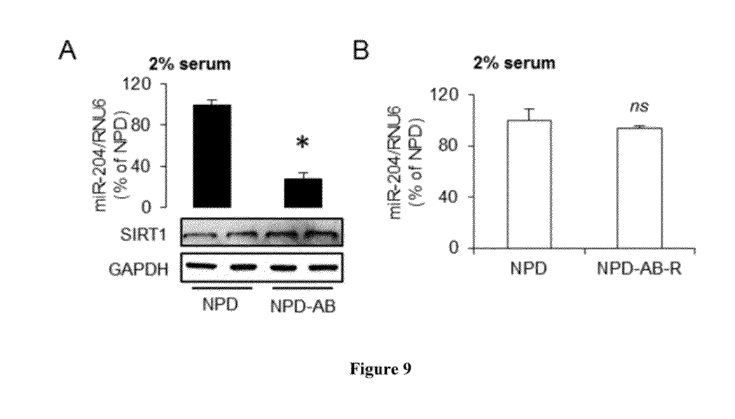

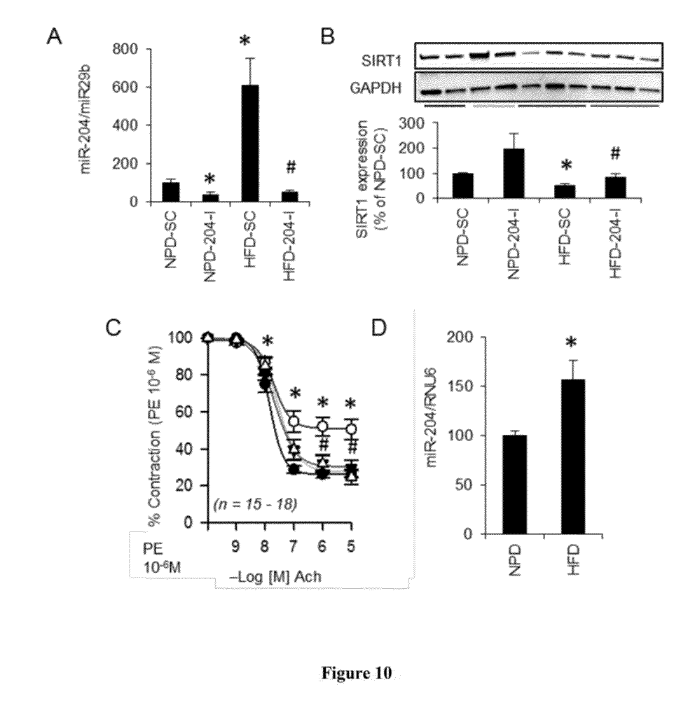

Finally, the role of systemic factors in gut flora-mediated regulation of endothelial miR-204 and SIRT1 was assessed. Addition of serum from mice whose microbiome was suppressed with antibiotics to endothelial cells resulted in decreased miR-204 and upregulation of SIRT1 (FIG. 9A). In contrast, serum from mice in which antibiotics were discontinued had no effect on endothelial miR-204 (FIG. 9B).

In sum, these findings reveal a gut flora-dependent mechanism for governing vascular function in which vascular miR-204 impairs endothelium-dependent vasorelaxation by downregulating endothelial SIRT1. Thus, strategies to manipulate the gut flora or vascular miR-204 may prove to be of therapeutic value for vascular diseases promulgated by endothelial dysfunction.

### Methods

Animals:

All animal experiments were approved by Institutional Animal Care and Use Committee and were carried out according to NIH guidelines. 10 week old male GFM and PFM were procured from Taconic, New York, USA. Endothelial-specific SIRT1 knockout (eSIRT1−/−) were generated by crossing SIRT1flox/flox mice with VE-Cadherin-Cre mice. Mice were given either normal pellet diet (NPD) or high-fat diet (HFD, 8-12 weeks). Some mice, housed separately, were given water supplemented with antibiotics (Metronidazole-1g/L, Ampicillin-1g/L, Neomycin-1g/L and Vancomycin-0.5g/L) for a period of six weeks to suppress the gut microbiota. To re-colonize the gut flora, antibiotic water was replaced with normal water and mice were mixed with the larger colony for a period of two weeks. The HFD is an adjusted calorie diet that provides 42% calories from fat (TD.88137, Harlan). The ALZET 2006 osmotic pups containing oligonucleotides (scrambled control or miR-204 inhibitor (miR-204-I)) were aseptically implanted in C57B1/6 mice kept on either NPD or HFD. The mini-osmotic pumps were designed to deliver oligonucleotides at the rate of 0.15 μl/hr, and each mouse received oligonucleotides at the dose of ˜0.7mgkg-1day-1 for a period of 6 weeks.

RNA Isolation and RT-PCR:

RNA was isolated using Qiazole/Trizole as per manufacturer's instructions. The concentration and purity of RNA samples were determined by Nanodrop. MiRs were converted to cDNA using by qScript™ miRNA cDNA synthesis kit (Quanta Biosciences, Gaithersburg, Md., USA). Real-time PCR was performed using Brilliant II SYBR Green qRT-PCR kit. The forward primers and universal reverse primer for miRs were procured from Quanta Biosciences, Gaithersburg, Md., USA. RNU6 or SNORD47 was used as internal control. RT-PCR of SIRT1, TRPM3 and GAPDH were performed using Brilliant II SYBR Green RTqPCR kit. GAPDH was used as internal control. The sequence of primers, miR mimics, miR-inhibitor and probe is provided in Table 1.

TABLE 1
Sequence of primers, mature miRs, detection probe, target protector and miR-mimic/inhibitor used
in the study.

| mRNA | Forward | Reverse |
|---|---|---|
| SIRT1 (mouse) | 5′-AAT GCT GGC CTA ATA GAC TTG | 5′-CCG TGG AAT ATG TAA CGA TTT G G-3′ |
| | CA-3′ (SEQ ID NO: 1) | (SEQ ID NO: 2) |
| TRPM3 (mouse) | 5′-ACC CCG TCA AGT AGT G-3′ (SEQ ID | 5′-CCC CAA AGT TGG CGT-3′ (SEQ ID NO: 4) |
| | NO: 3) | |
| GAPDH (mouse) | 5′-GGC AAA TTC AAC GGC ACA GT-3′ | 5′-CGC TCC TGG AAG ATG GTG AT-3′ (SEQ |
| | (SEQ ID NO: 5) | ID NO: 6) |
| GAPDH (human) | 5′-ATG ACA TCA AGA AGG TGG TG -3′ | 5′-CAT ACC AGG AAA TGA GCT TG-3′ (SEQ |
| | (SEQ ID NO: 7) | ID NO: 8) |
| eNOS (mouse) | 5′-GAA GGG AAG TGC AGC AAA GG-3′ | 5′-CAG AGA TCT TCA CTG CAT TGG CTA-3′ |
| | (SEQ ID NO: 9) | (SEQ ID NO: 10) |

| miRNA | Mature sequence |
|---|---|
| miR-204-5p | 5′-UUC CCU UUG UCA UCC UAU GCC U-3′ (SEQ ID NO: 11) |
| miR-29b | 5′-UAG CAC CAU UUG AAA UCA GUG UU-3′ (SEQ ID NO: 12) |
| miR-145-5p | 5′-GUC CAG UUU UCC CAG GAA UCC CU-3′ (SEQ ID NO: 13) |
| miR-148a-3p | 5′-UCA GUG CAC UAC AGA ACU UUG U-3′ (SEQ ID NO: 14) |
| miR-23b-3p | 5′-AUC ACA UUG CCA GGG AUU ACC-3′ (SEQ ID NO: 15) |
| miR-9-5p | 5′-UCU UUG GUU AUC UAG CUG UAU GA-3′ (SEQ ID NO: 16) |
| miR-122-5p | 5′-UGG AGU GUG ACA AUG GUG UUU G-3′ (SEQ ID NO: 17) |
| Small mRNAs | Sequence |
| RNU6 | 5′- GUG CUC GCU UCG GCA GCA CAU AUA CUA AAA UUG GAA CGA UAC AGA |
| | GAA GAU UUA GCA UGG CCC CUG CGC AAG GAU GAC ACG CAA AUU CGU GAA |
| | GCG UUC CAU AUU UUU-3′ (SEQ ID NO: 18) |
| SNORD47 | 5′-AAC CAA UGA UGU AAU GAU UCU GCC AAA UGA AAU AUA AUG AUA UCA |
| | CUG UAA AAC CGU UCC AUU UUG AUU CUG AGG UU-3′ (SEQ ID NO: 19) |
| Modulators | Sequence |
| miR-204-M | 5′-UUC CCU UUG UCA UCC UAU GCC U-3′ (SEQ ID NO: 11) |
| miR-204-I | 5′-AGG ATG ACA AAG GGA-3′ (SEQ ID NO: 20) |
| Scrambled control | 5′-ACG TCT ATA CGC CCA-3′ (SEQ ID NO: 21) |
| miR-204 probe | 5′-Dig-N-AGG CAT AGG ATG ACA AAG GGA A-N-Dig-3′ (SEQ ID NO: 22) |

NanoString nCounter miRNA Expression Assay, Data Analysis and Selection of Internal Control for RT-PCR:

100 ng of total RNA was used as input for the nCounter sample preparation reactions. All samples were prepared according to manufacturer's instructions. Briefly, a unique oligonucleotide was tagged to the miRNAs for the specific and sensitive detection by nCounter analysis system. After sample preparation nCounter analysis system was used to obtain miRNA expression data. The system is based on target-specific probe pairs that are hybridized to sample in solution. A reporter probe carries the fluorescent signal while capture probe allows the complex to be immobilized for data collection. The protocol does not include any amplification steps. The panel was designed to screen 578 mouse miRs, 33 viral miRs, 8 negative controls and a dilution series of 6 positive controls. NonoString nCounter raw data was normalized for lane-to-lane variation by six dilution series spike positive controls. To eliminate false positive results the mean and standard deviation of eight negative controls in each lane were determined. The sum of mean and 2× standard deviation was deducted from the raw value of individual miRNAs count. Global miR expression was used to normalize individual miR expression. The heat map for expression of miRs was prepared using GenePattern (http://genepattern.broadinstitute.org/gp/pages/index.jsf). In absence of universally accepted internal control for normalization of miR expression analysis by RT-PCR, we identified miR-29b as an internal control as its expression best resembled global miR expression.

In-Vitro and Ex-Vivo Transfection of miR-204 Mimic, Mir-204 Inhibitor and Scrambled Negative Control:

MiR-204-5p mimic, miR-204-5p inhibitor and scrambled control were purchased from Ambion, USA. MiR-204 mimic/inhibitor or scrambled negative control was incubated with Lipofectamine2000 (InVitrogen) at room temperature for 20 min before adding to cells in OPTI-MEM (InVitrogen). Four hours later the medium was replaced by fresh medium and cells cultured for an additional specified period of time. Ex vivo transfection of miR-204 mimic/miR-204-1/scrambled control into mouse thoracic aorta was performed using oligofectamine (Invitrogen). Oligos were incubated with oligofectamine at room temperature for 20 minutes before adding to aorta in OPTI-MEM (Invitrogen). The aorta was kept in transfection mixture overnight and OPTI-MEM medium and then replaced by EGM-2.

Vascular Reactivity:

Thoracic aortas of mice were used. The animals were rapidly euthanized by CO2 inhalation. The aorta was carefully dissected, rapidly removed, and placed in ice-cold oxygenated Krebs-Ringer bicarbonate solution. The vessel was carefully cleared of loose connective tissue and cut into 5-10 1.5 mm rings. A single ring from each mouse was suspended between two wire stirrups (150 μm) in a 12.5-ml organ chambers of a four-chamber myograph system (DMT Instruments) in 5 ml Krebs-Ringer (95% 02-5% CO2, pH 7.4, 37° C.). One stirrup was connected to a three-dimensional micromanipulator, and the other to a force transducer. The mechanical force signal was amplified, digitalized, and recorded (PowerLab 8/30). All concentration-effect curves were performed on arterial rings beginning at their optimum resting tone. This was determined by stretching arterial rings at 10 min intervals in increments of 100 mg to reach optimal tone (˜500 mg). One dose of KCl (60 mM) was administered to verify vascular smooth muscle viability. Cumulative dose-response curve for phenylephrine (PE) (10-9-10-5 M) was obtained by administering the drug in log doses. Endothelium-dependent and -independent vasodilatation was determined by generating dose-response curves to acetylcholine (ACh 10-9-10-5 M) and sodium nitroprusside (SNP 10-9-10-5 M), respectively on PE (10-6 M) induced pre-contracted vessel. Vasorelaxation evoked by ACh and SNP was expressed as percent relaxation, determined by calculating percentage of inhibition to the pre-constricted tension. NO bioavailability was measured physiologically by determining increase in the contractile response to NOS inhibition (L-NAME10-4M) in rings pre-constricted with PE (10-6M). Aortic rings were pre-incubated with recombinant Wnt3a (100 ng/ml) for 24 h.

Immunoblotting and Immunoprecipitation:

Protein samples were resolved on 10-12% SDS-PAGE, transferred to nitrocellulose membrane and analyzed with antibodies against SIRT1, p53, Ac-p53, β-actin and GAPDH. The antigen-primary antibody complexes were incubated with horseradish-peroxidase (HRP) conjugated secondary antibodies and visualized by western blotting luminol reagent (Thermo USA). Images were captured and quantified using Image Lab (BioRad, USA) software, and intensity values were normalized to β-actin or GAPDH. Tissue lysate was pre-cleared by incubating with IgG and Protein A/G plus-Agarose for 90 minutes.

Histological Processing, Immunohistochemistry and Immunofluorescence:

The tissues were stored in 10% formalin. Paraffin blocks were prepared. For immunohistochemical examinations 5 μm thick sections were prepared from paraffin blocks. Antigens were retrieved by heating (95° C., 20 minutes) in citrate buffer (10 mM). After incubation with primary antibodies the sections were incubated with polyvalent biotinylated goat anti-rabbit secondary antibody and streptavidin peroxidase (STV-HRP) system to amplify the signals, followed by detection with diaminobenzidine (DAB) as a chromogen. Slides were counterstained with hematoxylin, dehydrated with alcohols and xylene and mounted in DPX. For immunofluorescence the primary antibody antigen complex was probed with fluorescent tagged probes. Images were captured by charged coupled device (CCD) camera attached with the Olympus microscope (Model BX61) or Zeiss confocal microscope (Model 710).

In-Situ Hybridization:

Following deparaffinization and rehydration, aortic sections (6 μm) were treated with proteinase K (10 μg/ml, 5 minutes, 37° C.). The sections were dehydrated using ethyl alcohol and incubated with pre-hybridization buffer followed by hybridization mixture containing double DIG tagged miR-204-5p probe (36 hrs, 57° C.). The sections were washed with sodium saline citrate and probed with alkaline phosphatase (AP) conjugated Anti-DIG FAB (18 hrs, 4° C.), followed by detection with BCIP/NBT as a chromogen. Slides were counterstained with Nuclear Fast Red, dehydrated with alcohol and mounted in DPX. Images were captured by CCD camera attached with the Ziess microscope (Model BX61).

Cell Culture:

HUVECs and MASMCs were purchased from Clonetics (San Diego, Calif. USA) and cultured in endothelial growth medium (EGM-2, Lonza, Walkersville, Md. USA) and were used until passage 10. To examine the effects of mouse serum, instead of FBS pooled serum from the specific group of mouse was included. MASMCs were grown in equi-volume mixture of DMEM and F-12 media containing 10% FBS.

Statistical Analysis:

Statistical analysis was performed using SPSS (Version 17.0) statistical software. Significance of difference between two groups was evaluated using t-test. For multiple comparisons, one way ANOVA was used and post-hoc analysis was performed with Tukey's test. Results were considered significant if P values were <0.05.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of treating endothelial dysfunction, cardiovascular disease and/or atherosclerosis in a mammal, comprising administering an effective amount of a micro-RNA-204-5p inhibitor or a vector comprising an expression cassette comprising a promoter operably linked to a nucleic acid encoding a mircoRNA-204-5p inhibitor to the mammal.

2. The method of claim 1, wherein the inhibitor reduces microRNA-204-5p expression or function.

3. The method of claim 2, wherein microRNA-204-5p expression and/or function is reduced by at least 10%.

4. The method of claim 1, wherein the miRNA-204-5p inhibitor increases SIRTUIN1 (SIRT1) expression in a cell in the mammal, as compared to SIRT1 expression in a cell in a mammal that was not administered the micro-RNA-204-5p inhibitor.

5. The method of claim 1, wherein the miRNA-204-5p inhibitor increases endothelial nitric oxide synthase (eNOS) expression in a cell in the mammal, as compared to eNOS expression in a cell in a mammal that was not administered the micro-RNA-204-5p inhibitor.

6. The method of claim 1, wherein the miRNA-204-5p inhibitor comprises an oligonucleotide comprising 4 to 7 nucleotides that are complementary to miRNA-204-5p seed sequence 5′-UCCCUUU-3′ (SEQ ID NO:24).

7. The method of claim 1, wherein the miRNA-204-5p inhibitor comprises an oligonucleotide comprising a sequence having at least about 70% complementarity to miRNA-204-5p seed sequence 5′-UCCCUUU-3′ (SEQ ID NO:24).

8. The method of claim 1, wherein the miRNA-204-5p inhibitor comprises an oligonucleotide comprising a sequence having at least about 70% complementarity with miRNA-204-5p (5′-UUCCCUUUGUCAUCCUAUGCCU-3′ (SEQ ID NO:11)), at least about 70% sequence identity to miRNA-204-I (5′-AGGATGACAAAGGGA-3′ (SEQ ID NO:20)), or at least about 70% complementarity to miRNA-211 (5′-UUCCCUUUGUCAUCCUUCGCCU-3′ (SEQ ID NO:25)).

9. The method of claim 6, wherein the oligonucleotide is between about 18 to about 25 nucleotides in length.

10. The method of claim 6, wherein the oligonucleotide is chemically modified.

11. The method of claim 1, wherein the miRNA-204-5p inhibitor comprises an miRNA sponge, or a small molecule inhibitor.

12. The method of claim 1, wherein the vector is a plasmid or a viral vector.

13. A method of decreasing dysfunction in an endothelial cell that expresses micro-RNA-204-5p, comprising contacting the cell in vivo or in vitro with a micro-RNA-204-5p inhibitor.

14. A method of identifying a miRNA-204-5p inhibitor comprising
(a) comparing the level of miRNA-204-5p expression in an endothelial cell(s) before and after exposure to a small molecule, wherein the small molecule is a miRNA-204-5p inhibitor if the level of miRNA-204-5p expression is decreased in the endothelial cell(s) after exposure to the small molecule, or
(b) measuring a first level of miRNA-204-5p expression in an endothelial cell(s); contacting the endothelial cell(s) with a small molecule; measuring a second level of miRNA-204-5p expression in the endothelial cell(s); and comparing the first level of miRNA-204-5p expression with the second level of miRNA-204-5p expression, wherein, the small molecule is a miRNA-204-5p inhibitor if the second level of expression is decreased as compared to the first level of expression.

15. The method of claim 14, further comprising comparing SIRT1 expression in the endothelial cell(s) before and after exposure to the small molecule.

16. The method of claim 15, wherein the level of SIRT1 expression is increased in the endothelial cell(s) after exposure to the small molecule.

17. The method of claim 14, wherein the endothelial cell(s) was exposed to serum from an animal fed a high fat diet prior to exposure to the small molecule.

18. The method of claim 14, further comprising measuring a first level of SIRT1 expression in the endothelial cell(s) prior to the cell(s) being exposed to the small molecule; measuring a second level of SIRT1 expression after the cell(s) is exposed to the small molecule; and comparing the first level of SIRT1 expression with the second level of miRNA-204-5p expression.

19. The method of claim 18, wherein the second level of SIRT1 expression is increased as compared to the first level of SIRT1 expression.

20. The method of claim 14, further comprising contacting the endothelial cell(s) with serum from an animal fed a high-fat diet prior to measuring the first level of miRNA-204-5p expression.

21. The method of claim 14, wherein the level of miRNA-204-5p expression is measured using an activatable sensor oligonucleotide.
